(19) United States
(12) Patent Application Publication
(10) Pub. No.: US 2006/0247489 A1
(43) Pub. Date: Nov. 2, 2006

(54) Altered states of consciousness in virtual reality environments

(75) Inventors: Colin Richard Carbis, Victoria (AU); Joseph John Mastropaolo, Victoria (AU)

Correspondence Address: BACON & THOMAS, PLLC, 625 SLATERS LANE, FOURTH FLOOR, ALEXANDRIA, VA, 22314, US

(73) Assignee: Virtual Medicine Pty Ltd., Victoria (AU)

(21) Appl. No.: 10/551,423

(22) PCT Filed: Mar. 22, 2004
(86) PCT No.: PCT/AU04/00348

§ 371 Date: Jul. 5, 2006

(30) Foreign Application Priority Data
Apr. 1, 2003 (AU) 2003901669

(51) Int. Cl. A61M 21/00

(52) U.S. Cl. 600/27

(57) ABSTRACT

A device which induces altered states of consciousness by hypnosis, guided imagery, mediation or relaxation in a virtual reality environment. The embodiment discloses placing an audio visual headset on a subject, connecting this to a compact portable DVD player and then playing a short DVD movie so that the subject is placed into a relaxed state and ultimately drawing them into a deep state of hypnosis.

### 5 BACKGROUND OF THE INVENTION

Part 1—Hypnosis

According to Blakiston's Gould Medical Dictionary (1984), hypnosis may be defined as a state of altered consciousness, sleep, or trance; induced artificially in a subject by means of verbal suggestion by the hypnotist or by the subject's concentration upon some object; characterized by some extreme responsiveness to suggestion made by the hypnotist. The degree of the hypnotic state may vary from mild increased suggestibility to that comparable to surgical anesthesia. Hypnotherapy is defined as (a) the treatment of disease by means of hypnotism, and (b) the induction of sleep for therapeutic purposes.

Hypnosis procedures, using similar techniques to those practiced today, were described more than 3,000 years ago in the Ebers Papyrus. The modern era of hypnosis began in Vienna in 1773 when a medical student, Franz Anton Mezmer (1734-1815), presented his doctoral thesis, entitled “De Planetarium Influx”. Mesmer's thesis referred to a strange magnetic fluid in the human body, and he claimed that disease occurred when magnetic fluids in the body were depolarized. Mesmer developed an unorthodox treatment protocol, where he repolarised his patient's magnetic fluid using a bath filled with water and iron filings. In later years, Mesmer found that he could achieve the same results without his magnetic bath and other paraphernalia. He claimed that he himself possessed a magnetic force, which he described as animal magnetism.

According to Mesmer, animal magnetism achieved unparalleled success in the treatment of human disease, and, although testimonials from his patients supported his claims, he was accused of charlatanry. In 1784, Louis XVI of France appointed a commission to investigate Mesmer's technique and claims to success. The commission concluded that there was no such thing as animal magnetism, and, although Mesmer's cures seemed to work, they were scientifically groundless and therefore unacceptable. Mesmer's practices were described as sham, and, because of ridicule from his peers, he retired to Switzerland where he died in 1815.

In 1837, during the British rule of India, a Scottish doctor named James Esdaile used hypnosis to anaesthetize patients, and he performed several hundred painless surgical procedures—including amputations. Fatal surgical shock or postoperative infection occurred in only 5% of Esdaile's patients. In comparison, Esdaile's contemporaries performed surgical procedures without hypnosis or chemical anesthesia, and they reported fatal surgical shock or postoperative infection in about 50% of their patients (Zach 1980).

In the 1920's, Emile Coue demystified hypnosis and established hypnotherapy as a credible, reliable therapeutic technique, demonstrating how simple suggestions can bring health and well being to the ill. In the present day environment, hypnosis is a recognized medical technique used, for example, in obstetrics, surgery, anesthesia, pediatrics, psychiatry, nursing and dentistry (Gonsalkorale 1996).

Part 2—Virtual Environments

Virtual environments are a new technology, and there is some debate in the literature concerning what constitutes a virtual environment. In this patent, a virtual environment is considered to be any two dimensional or three dimensional display device that shows objects, still pictures or moving pictures for the purpose of creating a virtual world/virtual environment for the user of the device. Display devices would typically include head mounted displays or wide field-of-view displays such as projection screens.

A wide field-of-view display (e.g. on a projection screen) or a head mounted display is utilized to give the user an illusion of spatial immersion, or presence, within the virtual environment. A virtual environment display that is fixed in space is referred to as partially immersive virtual reality. A fully immersive virtual reality environment utilizes a head mounted display, with a head position sensor to control the displayed images so they appear to remain stable in space when turning the head or moving through the virtual environment.

Part 3—Hypnosis in Virtual Environments

Hypnosis in a virtual environment has not been described in the literature.

### 6 SUMMARY OF THE INVENTION

According to the invention, hypnosis, guided imagery or meditation in a virtual environment is used for, but not limited to, relaxation, meditation, guided imagery, childbirth, anesthesia, spiritual development, sports training, skill development, motivation and the treatment or management of pain, somatic and/or psychosomatic diseases, physical trauma, grief, phobias, wound dressing changes and mood and behavioral disorders.

Hypnosis, guided imagery or meditation in a virtual environment comprises all or some of the following components. The following list is offered for illustration purposes, and they are not intended to limit or define the invention in any manner.
Visual images that may include two-dimensional or three-dimensional motion pictures, still pictures, cartoons, computer graphics and various moving or still objects.
Auditory stimuli that may include, musical instruments, sounds from the human voice such as chanting and humming, environmental sounds such as animal calls, wind, rain, waterfalls and thunder and mechanical noises such as car engines and pumps.
Verbal communications that may include suggestions and/or directions for relaxation, meditation, guided imagery, childbirth, anesthesia, spiritual development, sports training, skill development, motivation and the treatment or management of pain, somatic and/or psychosomatic diseases, physical trauma, grief, phobias, wound dressing changes and mood and behavioral disorders.
Any two dimensional or three dimensional display device that shows objects, still pictures or moving pictures for the purpose of creating a virtual world/virtual environment. A wide field-of-view display (e.g. on a projection screen) or a head mounted display that provides the user with an illusion of spatial immersion, or presence, within the virtual environment is a typical display device
A head position sensor that can be fixed on a head mounted display, which controls displayed images so they appear to remain stable in space when the user turns their head or moves through the virtual environment.
A microphone or input device directed into the virtual environment, which can be used by qualified hypnotherapists to provide specific post induction counseling.
A microtechnology DVD/VCD player, so the unit can be packaged into a portable, shockproof briefcase.

In literature that describes the use of hypnosis, the patient is guided into an altered state of consciousness by the voice of a hypnotherapist. The induction process may be assisted by the use of a bright stationary or moving object, such as a candle, a light or a watch. The literature also describes the use of video inductions/hypnosis on television and projection screens (Miller et al., 1992). Hypnosis, guided imagery or meditation in a fully or partially immersive virtual reality environment has not been described in the literature previously.

The main features that distinguish standard techniques of hypnosis and virtual reality from hypnosis in a virtual reality environment are summarized below.
(a) In a virtual environment, hypnosis is achieved by the use of recorded images, verbal communication and non-verbal sounds, and the depth of relaxation and/or the trance state is not dependant on the hypnotherapy skills of the person operating the device. In practitioner-guided hypnotherapy, the depth of relaxation and/or the trance state is dependant on the skills of the hypnotherapy practitioner.
(b) In a virtual environment, repetitive rhythms of scene changes and non-verbal sounds are used to enhance and support verbal suggestions that lead to eye fatigue and eye closure. In practitioner-guided hypnotherapy, eye fatigue and eye closure are limited by patient compliance with verbal suggestions offered by the practitioner.
(c) In a virtual environment, visual images including, but not limited to, hypnotic spirals are used to cause eye fatigue and surreal perceptions that enhance and support verbal suggestions for eye closure. In practitioner guided hypnotherapy, eye fatigue and surreal perceptions are limited by the patient's ability to create mental images.
(d) In a virtual environment, visual and auditory stimuli are coordinated with and augment verbal suggestions, making it difficult for the user to avoid compliance with verbal suggestions that lead to relaxation and eye closure. In practitioner-guided hypnotherapy, the patient has to concentrate on the practitioner's voice, and, because unrelated noise is not coordinated with verbal suggestions, it becomes a distraction that reduces the clinical efficacy of hypnosis therapy.
(e) In a virtual environment, the users thoughts are focused on a variety of visual and non-verbal stimuli, which reduces conscious, critical analysis of verbal suggestions. By reducing critical analysis of verbal suggestions at the conscious level, the user is more likely to accept therapeutic messages at the sub-conscious level; thereby modifying belief systems that perpetuate undesirable physiological outcomes. In practitioner-guided hypnotherapy, the conscious mind is focused on the human voice, where non-verbal messages associated with intonation, voice texture, pauses, accents, imperfections (e.g. lisps etc.) and other anomalies will encourage critical analysis of verbal suggestions and the character of the practitioner. Critical analysis of verbal suggestions or the practitioner will reduce the clinical efficacy of therapy.
(f) In practitioner-guided hypnotherapy, the patient invariably conducts a character analysis of the hypnotherapist, and the efficacy of therapy is limited by the patient's appraisal of the practitioner and the amount of trust and rapport established between them. The practitioner may need several hours with a patient to build enough rapport for successful hypnosis, and, by that time, the patient may have lost confidence in hypnotherapy for the treatment of their condition. In a virtual environment, therapy is interfaced with technology, so the user is not confronted by inherent desires for character analysis and establishing high-level trust and rapport with the person operating the equipment
(g) In a virtual environment, metaphors are, or can be, coordinated with visual and non-verbal stimuli to create theatrical surrealism, which immerses the user in a role-play environment where therapeutic suggestion is linked with metaphoric narrative. In surreal theatrical environments created by virtual reality, lessons embedded in metaphoric narrative are more likely to evade conscious, critical analysis, and be assimilated in belief structures at subconscious levels; providing alternative pathways for inappropriate thought. In practitioner-guided hypnotherapy, theatrical surrealism and effective role-play are more difficult to achieve, so the lessons from metaphoric narrative may be lost in critical analysis at conscious or semi-conscious levels.
(h) In scientific literature, virtual reality is described as an interactive environment (Riva 1997), where the user is:
   Not guided to use their imagination for the purpose of creating images in their mind.
   Not encouraged to relax and close their eyes.
   Not encouraged to dissociate their mind from their body.
   Not encouraged to eliminate all conscious thought in the virtual environment.
      Hypnosis in a virtual environment is a novel procedure, not previously described in the literature, where the user is:
   Guided to use their imagination for the purpose of creating images in their mind.
   Encouraged to relax and close their eyes.
   Encouraged to dissociate their mind from their body.
   Encouraged to eliminate all conscious thought in the virtual environment.

The significance of the above features is summarized below.
(a) In practitioner-guided hypnotherapy, patients may feel uncomfortable or vulnerable closing their eyes. Relaxation, therefore, is difficult, and they fail to reach a deep trance state that is optimal for hypnotic suggestion.
(b) In a virtual environment, an audio visual induction method can be delivered repeatedly in a consistent, reliable manner—eliminating variations in hypnotic suggestions, metaphors, mood, voice inflections, mannerisms and other uncontrolled variables of the hypnotist and the patient.
(c) Altered states of consciousness induced by audiovisual presentations are economical, safe and time efficient. People involved in patient care, providing hypnosis in a virtual environment, do not require expert knowledge of the process.
(d) Some patients develop an emotional attachment to their therapist, and these feelings may compromise the patient/therapist relationship. In a virtual world, the patient/therapist relationship is more distant, so emotional attachments to the therapist are less likely to occur.
(e) In a virtual environment, each therapeutic application requires a separate DVD, VCD or video, so the clinical utility of each product can be evaluated. In practitioner-guided hypnotherapy, hypnotic suggestions, metaphors, voice gestures, verbal content, voice intonation etc. is highly variable between therapists, so, compared to virtual hypnosis, the outcome is also more variable, making it difficult to evaluate or control a given procedure.
(f) On a DVD, verbal suggestions are fully rehearsed and repeated until all aspects of the recorded voice are optimized for hypnotherapeutic efficacy. In practitioner-guided hypnotherapy, the practitioner's voice may contain flaws that jolt the patient into conscious thought; thereby reducing the efficacy of the hypnotherapy.

### 7 DETAILED DESCRIPTION OF THE INVENTION

Hypnosis in a virtual environment has been used to treat patients in Melbourne hospitals for trial purposes and to establish proof of principle. The following case studies are offered for illustration purposes, and they are not intended to limit or define the invention in any manner.

Case Study 1—Pain Management in Cancer

Introduction and Patient Background

The patient was a 36year-old female with cancer who developed Hodgkins Lymphoma at 19 years of age with subsequent lung metastases. Problems associated with her condition are highlighted in the following bullet points.
The malignancy was aggressively treated with cardiotoxic, chemotherapy drugs. The chemotherapy drugs caused cardiac insufficiency and, eventually, she needed a heart transplant.
The patient had a tracheotomy tube inserted because muscles in her neck and chest were so weak that she had extreme difficulty removing phlegm and saliva from her lungs by coughing. Sputum and saliva were removed via the tracheotomy tube. The tracheotomy tube interfered with oral feeding, so a stomach tube was inserted to supply food.
The patient developed facial palsy, and lost control of muscles on the left side of her face. She was unable to open her left eye, and vision in her right eye was blurred. Blurred vision was partially corrected with glasses. Behind her left eye, she had extreme pain—refractory to chemical analgesia.
The patient had four intensive care admissions during the previous five months before hypnosis in a virtual environment.
The patient had spent most of her life in hospital, and lack of exercise caused severe musculoskeletal pain—particularly in her legs. She had difficulty sitting in an upright position, and was unable to walk.
   Materials and Methods

Virtual Hypnosis was provided to the patient using a portable brief case unit specifically designed by Virtual Medicine. The Virtual Hypnosis device consisted of a head mounted display and a micro-DVD player supplied by Mindflux, and a DVD called Virtual Analgesia (Rivers). The patient experienced relaxing country scenes, a hypnotic spiral, various non-verbal sounds, and verbal directions for relaxation and therapeutic suggestion. The patient's ability to hear or see anything outside the virtual environment was limited by confines of the head mounted display.

The patient was treated in the Alfred hospital oncology ward at 8 pm on three consecutive evenings. On the first meeting, the patient was asked to rate her pain experience on a scale from 0 to 10, where 0 refers to no pain and 10 refers to the most severe pain experience. The patient was made comfortable in her bed, and the head mounted display was fitted comfortably on her head. The DVD player was switched on, the audio levels were adjusted, the lighting was dimmed, and everyone left the room. During hypnosis in the virtual environment, the patient was checked at five-minute intervals to monitor for adverse events. When the session was completed, the head mounted display was removed, and the patient was asked to rate her pain experience. On the days that followed each session, nurses and family members were interviewed to assess behavioral changes, and the patient was asked to rate her pain experience for the day.

Results

Day 1: The patient could hear verbal and non-verbal sounds through the earphones, but, after the session, she indicated that visual images were absent. Apparently the glasses weren't properly adjusted on her head. Because of the tracheotomy, she couldn't speak and didn't ask to have the headset adjusted. She didn't know if visual images were meant to be part of the experience. On the following day, care nurses and family members did not observe behavioural changes.

Day 2: The head mounted display was correctly adjusted, and the patient saw visual images—as expected. The patient's sister commented on her relaxed appearance during the session, and said it was rare to see her look comfortable. During therapy, the patient was compliant with verbal suggestions, and she closed her eye and entered into a deep sleep/trance state at the time indicated by verbal suggestion in the DVD. The patient remained asleep when the DVD had finished, but she woke up when the head mounted display was removed. At the end of therapy, the patient was happy and smiling uncontrollably. Her facial expression had radically changed. The patient rated her pain experience at the end of the session (Table 1). On the following day, the care nurse said the patient was less demanding, easier to get along with all day, and required less attention and chemical analgesia from the nurses. Other nurses and family members noticed that the patient was more relaxed and less tense than usual.

Day 3: After the third treatment, the patient's self reported pain assessment for her leg muscles was zero. Hospital staff reported continued improvement in the patient's behavior on the following day. Treatment was discontinued after three sessions.

TABLE 1
Patient ratings for indicators of pain and emotional
state following Virtual Analgesia (VA) therapy.

| | Patient rating for pain | | | |
|---|---|---|---|---|
| | | Day 1 - | | |
| | | No visual | | |
| | Pre-VA | images | Day 2 | Day 3 |
| Post VA - leg muscles | 10 | 10 | 5 | 0 |
| Post VA - behind left eye | 8 | 8 | 6 | 7 |

Conclusion

Hypnosis in a virtual environment (using Virtual Analgesia—Rivers) was used on three consecutive evenings for the treatment of pain. On the first evening, the headset was not positioned correctly on the patient's head, and visual images could not be seen. Without visual images, the patient was not effectively immersed in a virtual environment, and she reported no change in her pain experience.

On the second and third evenings, the headset was correctly positioned, and the change in the patient's pain experience was highly significant. After the second session, leg muscle pain was reduced from 10 to 5 using the patient's own score rating, and after the third session the patient rated score was reduced from 5 to 0.

The patient had zero vision in her left eye, and blurred vision in her right eye, so she had limited ability to see visual images and become fully immersed in the virtual environment. Nonetheless, her self reported experience of leg muscle pain was reduced by 100%, and nursing staff commented favorably on behavioral/mood changes on the days following treatment.

Virtual Analgesia did not effectively manage eye pain, and only small improvements in patient rated scores for eye pain were reported after treatment. However, eyestrain, blurred vision, monocular vision and inability to focus are contraindications for treatment using hypnosis in a virtual environment, so ineffective pain management for eye pain was not unexpected in this patient.

Case Study 2—Anxiety Disorder in a Burns Patient

Introduction and Patient Background

On the 12^{th} of Oct. 2002, Muslim extremists exploded an incendiary device that destroyed the Sari club in Bali Indonesia. The Sari club was a popular destination for travelers, and the terrorists carefully planned the explosion to kill American, Australian and English tourists.

The patient described in this study was a 28-year-old Australian, visiting Bali with his local football team. He went to the Sari Club on his first night in Bali, and was standing about 10 meters away from the bomb when it exploded. The force of the blast hurled him several meters across the room amid a fireball that burned 60% of his total body surface area and ruptured tympanic membranes in both ears. After a short period in the Bali hospital, he was transferred to an Intensive Care Unit at a Melbourne Hospital. The patient was discharged from the hospital 43 days after the Bali bomb blast.

Materials and Methods

Virtual Hypnosis was provided to the patient using a portable brief case unit specifically designed by Virtual Medicine. The Virtual Hypnosis device consisted of a head mounted display and a micro-DVD player supplied by Mindflux, and a DVD called Virtual Analgesia (Rivers). The patient experienced relaxing country scenes, a hypnotic spiral, various non-verbal sounds, and verbal directions for relaxation and therapeutic suggestion. The patient's ability to hear or see anything outside the virtual environment was limited by confines of the head mounted display.

Virtual Hypnosis treatment commenced 17 days after the Bali bomb explosion. The patient was treated in the evenings at about 8.00 to 9.00 pm at the end of visiting hours. Prior to Virtual Hypnosis treatment, the patient was asked to rate his pain experience on a scale from 0 to 10, where 0 refers to no pain and 10 refers to the most severe pain experience. The patient was made comfortable on the hospital bed, and the head mounted display was fitted on the patient. The DVD player was switched on, the audio levels were adjusted, the lighting was dimmed, and everyone left the room. During hypnosis in the virtual environment, the patient was checked at five-minute intervals to monitor for adverse events. After treatment, the head mounted display was removed, and the patient was left undisturbed for the rest of the night (except for routine monitoring by nurses). The patient rated pain score was completed the following evening, so that relaxation and attention to therapeutic suggestions were not interrupted by the need to concentrate on interview questions. The treatment schedule was determined by patient and/or hospital staff requests.

Results

In the post admission period, the patient was withdrawn, refused radio and television installations and was agitated by nursing staff opening the curtains around his bed. He was uncooperative, irritated by nursing interventions, and had limited interaction with hospital staff.

In post admission interviews, he reported limited recall of post explosion events in Bali, although, he had strong memories about thoughts of imminent death during his efforts to escape the burning nightclub. He declined psychiatric consultation, refused to talk about events in Bali or recovery from his injuries, and he denied emotional distress.

Burns injuries were complicated by acute stress disorder (ASD), characterized by anxiety, tearful outbursts (particularly at the end of visiting hours), insomnia, panic attacks, depression, delayed mobilization and erratic mood swings. The patient described a strange feeling of not being himself, and his fear of being alone was reinforced by failure of the hospital call system.

Day sleep was excessive and night was characterized by insomnia and recurrent nightmares during intermittent periods of sleep. He often panicked when he woke at night, and screamed for help if there was no one in the room. For 20 to 30 minutes after waking, he experienced feelings of dread, tachycardia, hyperventilation, sweating, subjective feelings of suffocation and visions of the walls closing in on him. He had frequent nightmares about escaping the burning nightclub, and he thrashed around on the bed causing further damage to his wounds. During one nightmare, he fell off the bed, panicked and maneuvered himself behind the door, preventing hospital staff from entering the room. After this event, the hospital had someone seated outside his room 24 hours a day, to comfort him when he woke in panic. Exacerbations of panic episodes were associated with changes in staff, room allocations and unfamiliar views from hospital windows.

ASD was intransient to standard treatment methods, and symptoms related to this condition persisted for 19 days after the Bali explosion. The hospitals Pain Management Unit felt that recovery, analgesia requirements and pain experience were influenced by cognitive factors, and they believed the patient might benefit from Virtual Hypnosis therapy. The patient was skeptical about the benefits of Virtual Hypnosis, and in the pre-treatment interview he was short tempered and provided terse replies to all interview questions. Other indicators of ASD observed during the pre-treatment interview included anxiety, distress, grey pallor, inconsistent vocal tonation, depressed body language (particularly facial expressions) and difficulty maintaining eye contact. The patient was negative to contraindications for Virtual Hypnosis (i.e. epilepsy, schizophrenia and vision impairment) and potentially positive to hypnotic susceptibility (i.e. childhood sleepwalker).

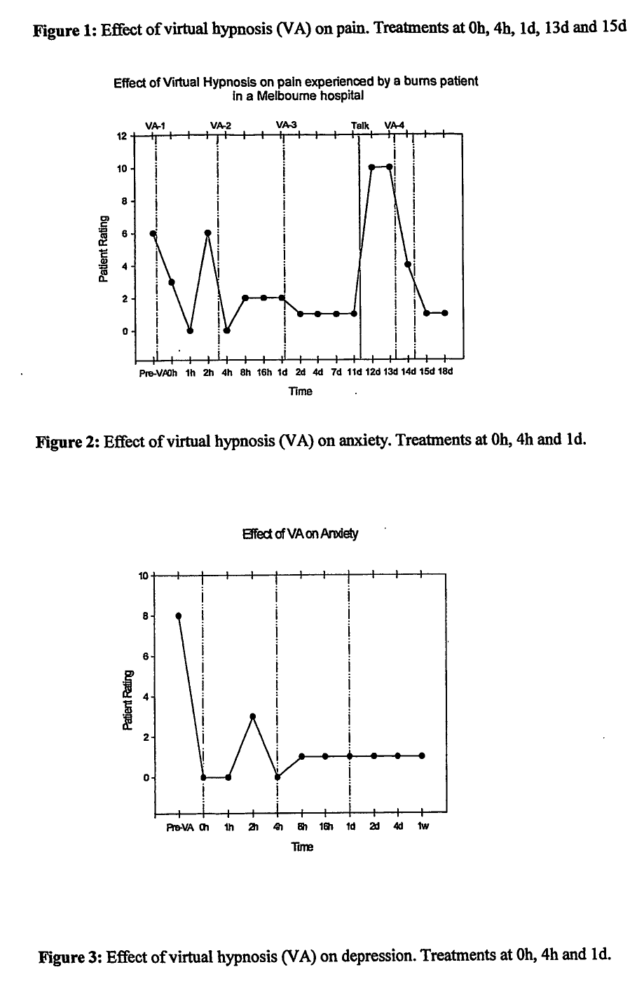

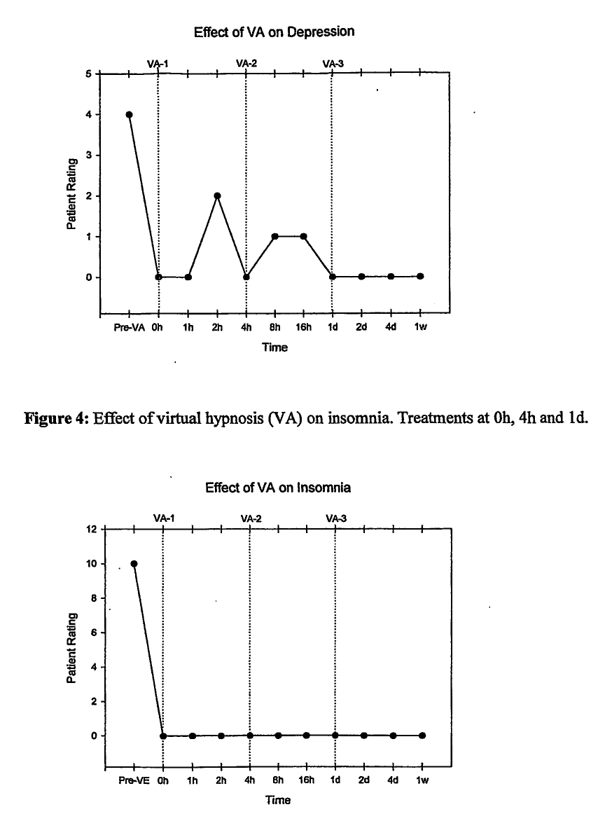

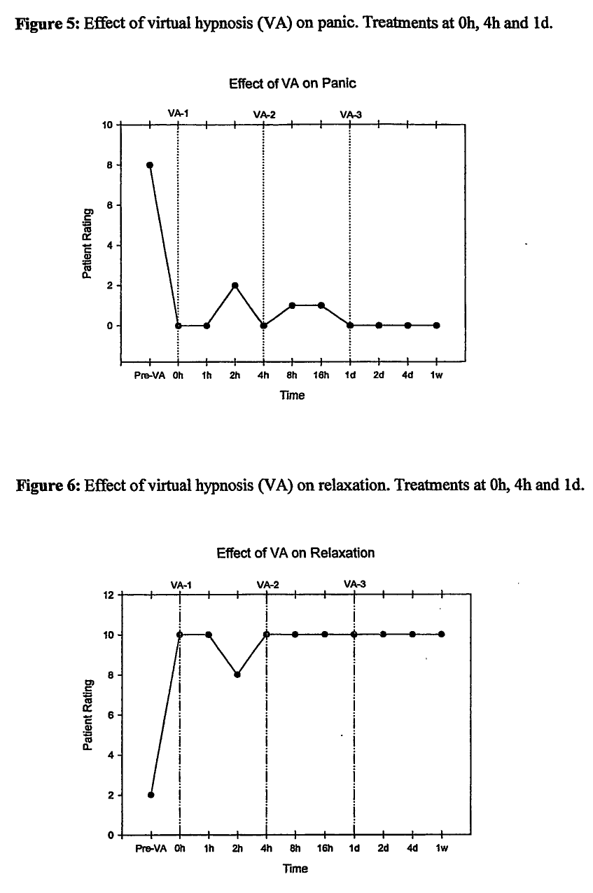

The patient slept for several hours, without nightmares or recall of dreams, after his first day of treatment (19 days post explosion) with Virtual Hypnosis. His grey pallor disappeared, analgesia requirements were reduced and dread, tachycardia, hyperventilation, sweating and other panic related symptoms were absent when he woke the following morning. The following day, the patient described his Virtual Hypnosis experience, where he imagined that pain was associated with a rabbit in his garden. He imagined the rabbit was getting smaller, and his pain gradually diminished as the rabbit faded from view. The patient was asked to rate his pain experience, insomnia, anxiety, depression, panic, relaxation and sadness (FIGS. 1-7).

The patient was fearful that nightmares and insomnia might return without treatment, so he asked for Virtual Hypnosis therapy the following evening. On the third evening (21 days post explosion), he felt confident to try night sleep without Virtual Hypnosis treatment, and he successfully slept through the night without nightmares or clinical signs of panic the following day. Staff and patient reports indicate that analgesia requirements and clinical signs of ASD progressively improved in the post Virtual Hypnosis period.

Thirty-two days after the Bali explosion, the patient overheard nursing staff making negative comments about his wounds. During sleep that evening, his nightmares returned, and he woke screaming in fear of losing his legs. Once again, he had visions of the walls caving in, and experienced dread, tachycardia, hyperventilation and tightness in his chest. The patient tried to emulate his Virtual Hypnotherapy experience without success. Nursing staff requested further Virtual Hypnosis treatment for the following evening. After treatment, the patient slept through the night without nightmares, and symptoms of ASD were largely absent the following morning (FIG. 1-7). An abrupt transition to mobility occurred 34 days post explosion, and, for the remainder of his hospital stay, he made rapid progress without ASD related symptoms.

Discussion

ASD occurs in 12% of trauma survivors. Burns victims with even mild to moderate symptoms of ASD have difficulties with physical and psychological adjustments following their discharge from hospital, and their long-term adjustment phase is frequently complicated with co morbidities including posttraumatic stress disorder (PTSD), depressive disorders, adjustment disorder, affective disorder and substance abuse (Fauerbach et al., 1999). The most common types of trauma events that cause ASD include combat (Simms et al., 2002 and Glenn et al., 2002), natural disasters (Mc Farlane and Papay 1992), motor vehicle accidents (Blanchard et al., 1995), crime or injury (Resnick et al., 1993) and severe burn injuries (Fauerbach et al., 1997 and Taal and Faber 1998). In a two-year prospective evaluation of the relationship between ASD and PTSD, Harvey and Bryant (2000) found that 80% of trauma survivors diagnosed with ASD developed chronic PTSD. However, chronic PTSD may be avoided if ASD is treated with early intervention cognitive behavior therapy (Bryant et al., 2000).

In trauma patients, management of ASD and other psychosomatic illness is a secondary priority, which may not be addressed unless symptoms of the cognitive process impact on recovery from physical injuries. The patient described in this study fell from the hospital bed and compromised skin grafts during repeated panic episodes, so the treatment of cognitive issues developed a higher profile.

Patient anxiety was compounded with anger and frustration, which was often directed at hospital staff providing nursing interventions, counseling and psychiatric help. Patient frustration was also evident in the pre-Virtual Hypnosis interview, where body language and terse replies to interview questions demonstrated poor control of cognitive processes. In the pre-Virtual Hypnosis interview, practitioner guided hypnotherapy was considered highly unlikely to be effective, since the patient was agitated by discussion and all human contact. The patient was notably relieved when advised that everyone would leave the room, and he was instantly comforted by visual images in the virtual environment.

After the first night of Virtual Hypnosis treatment, the patient and hospital staff reported a highly significant reduction in ASD symptoms. Nightmares and insomnia were instantly controlled, and the patient enjoyed his first night of uninterrupted sleep after the Bali explosion. Panic attacks were absent when he woke the following morning, and requirements for burn pain analgesia were managed at reduced levels. Clinical signs of ASD were absent for 13 days post Virtual Hypnosis treatment, until the patient overheard nurses making negative comments about his skin grafts during routine dressing changes. The patient's catastrophic interpretation of the nurse's remarks manifested in depressive thinking, and the symptoms of ASD returned: i.e. severe panic episodes, nightmares, insomnia and a grossly elevated pain experience. The patient requested further treatment when he failed to emulate his previous Virtual Hypnosis experience. His previous success and positive approach to Virtual Hypnosis seemed to further enhance the efficacy of treatment (FIGS. 1-7).

In this study, Virtual Hypnosis controlled nightmares and insomnia, reduced analgesic requirements, reduced patient calls for nursing assistance, removed the need for round the clock surveillance, controlled patient anger and frustration, and assisted his transition to mobility. Most importantly, Virtual Hypnosis controlled anxiety, panic attacks and depressive thinking, which can result in chronic elevation of glucocorticoids like cortisol when cognitive disorders are not effectively managed. Chronic elevation of cortisol levels may cause immunosuppression, which may increase the likelihood of wound infections and decrease the rate of wound healing (Schrader 1996). According to studies by Bryant et al., 2000, the probability this patient will develop comorbidities, like PTSD, depressive disorders and substance abuse, is markedly reduced following his discharge from hospital, since Virtual Hypnosis effectively controlled ASD.

Case Study 3—Hypnosedation in Surgical Environments

Introduction and Patient Background

The patient was a 34year-old female in labor at the Francis Perry Hospital (Melbourne). Labor was induced with oxytocin, and the patient was anticipating normal vaginal delivery. After 10 hours of labor, the obstetrician decided that vaginal delivery was associated with unacceptable risks, and she recommended surgical intervention by Caesarian section. The obstetrician's comments were unexpected, and imminent surgery manifested in anxiety and emotional trauma for the patient and her family.

Materials and Methods

Virtual Hypnosis was provided to the patient using a portable brief case unit specifically designed by Virtual Medicine. The Virtual Hypnosis device consisted of a head mounted display and a micro-DVD player supplied by Mindflux, and a DVD called Virtual Analgesia (Rivers). The patient experienced relaxing country scenes, a hypnotic spiral, various non-verbal sounds, and verbal directions for relaxation and therapeutic suggestion. The patient's ability to hear or see anything outside the virtual environment was limited by confines of the head mounted display.

The patient was relocated from the labor ward to theatre. Additional anesthesia was not administered, since pain was adequately managed by epidural. The patient wanted the head mounted display removed as soon as possible after her baby's delivery.

During skin asepsis, fitting the head mounted display reduced the patient's visual and auditory contact with the surgical environment. Surgical procedures began about 15 minutes after virtual hypnosis commenced, and, by this stage, the patient was in a relaxed state.

Results

In theatre, Virtual Hypnosis markedly reduced anxiety, and surgery proceeded without incident. The head mounted display was removed postpartum, so the mother could have immediate contact with her baby. The cognitive adjustment from virtual environment to surgical environment was immediate, and the patient enjoyed full conscious awareness of her baby.

According to midwives attending the patient, post Caesarian recovery was about 2 days ahead of the average hospital expectations for perambulation, patient requested analgesia and psychological adjustment.

Discussion

Women frequently enter obstetric environments with a birthing plan, and unexpected changes to their schedule may cause stress and anxiety. Obstetricians invariably discuss pain management options with their patients, but anxiety and its plethora of physiological and psychological sequelae are frequently overlooked.

In this patient, pre-Caesarian anxiety was managed using Virtual Hypnosis, and when surgery commenced the patient was calm and relaxed. The virtual reality experience had flow on effects for the patient's partner, who was concerned by his wife's anxiety.

Midwifes attributed the patients rapid post Caesarian recovery to the management of anxiety using Virtual Hypnosis. Hospital costs are reduced by shorter hospital stays, reduced use of analgesics and lower demand for nursing.

Case Study 4—Treatment of Psychosomatic Disease (Muscle Pain)

Introduction and Patient Background

The patient was a 62-year-old male, employed as a middle manager in a high-stress, office environment He suffered from spasms and consistent muscle pain in his neck and shoulders, which, he said, was caused by work related stress. The patient supported this belief by describing pain remissions during holiday periods remote from his work environment.

Materials and Methods

Virtual Hypnosis was provided to the patient using a portable brief case unit specifically designed by Virtual Medicine. The Virtual Hypnosis device consisted of a head mounted display and a micro-DVD player supplied by Mindflux, and a DVD called Virtual Analgesia (Rivers). The patient experienced relaxing country scenes, a hypnotic spiral, various non-verbal sounds, and verbal directions for relaxation and therapeutic suggestion. The patient's ability to hear or see anything outside the virtual environment was limited by confines of the head mounted display.

Prior to Virtual Hypnosis treatment, the patient was asked to rate his pain experience on a scale from 0 to 10, where 0 refers to no pain and 10 refers to the most severe pain experience. The patient was made comfortable on his couch, and the head mounted display was fitted comfortably on his head. The DVD player was switched on, the audio levels were adjusted, the lighting was dimmed, and everyone left the room. During hypnosis in the virtual environment, the patient was checked at five-minute intervals to monitor for adverse events. When the session was completed, the head mounted display was removed, and the patient was asked to rate his pain experience 30 minutes, 12 hours and 24 hours after Virtual Hypnosis.

Results

The patient was drowsy for about 15 minutes after Virtual Hypnosis. His relaxed outlook and changes in vocal tone and intonation indicated that stress levels were markedly reduced. Postural changes were evident in the post treatment interview, and the patient rated pain scores were less than pre-Virtual Hypnosis levels (Table 1).

TABLE 2
Patient rated pain score for neck and shoulder muscle pain.

| | Patient rating for pain | | | |
|---|---|---|---|---|
| | | Day 1 - | Day 2 | Day 3 |
| | Pre-VA | 30 min | 12 hours | 24 hours |
| Post Virtual Hypnosis - | 6 | 0 | 0 | 2 |
| neck and shoulder pain | | | | |

Conclusion

In one session, Virtual Hypnosis (Virtual Analgesia—Rivers) produced complete pain remission in the patient's neck and shoulder muscles. At 24 hours, pain levels began to rise, but the patient rated pain score was still less than half the pre-treatment score.

Physical trauma and autoimmnune diseases (e.g. arthritis and fibromyalgia) were excluded as possible causes for this man's condition, and his neck and shoulder pain seems to have psychosomatic origins manifested by the cognitive approach to his work environment.

Virtual Hypnosis treatment effectively controlled pain associated with this man's emotional response to unresolved issues in his work environment, and this study indicates that Virtual Hypnosis may have wider applications in the treatment of other psychosomatic diseases.

### 5 SEARCHES

A comprehensive literature search was conducted using Medline (1966-2003), and a dial index search on the “all patents” category.

Key words used in searches include hypnosis or virtual reality.

### 9 REFERENCES

Blakiston's Gould Medical Dictionary (1984), Fourth Edition, McGraw Hill Publishing Company, New York.

Blanchard, E. B., Hickling, E. J., Taylor, A. E. and Loos. W. (1995) Psychiatric morbidity associated with motor vehicle accidents. Journal of Nervous and Mental Disorders 183 (8): 495-504.

Bryant, R. A., Sackville, T., Dang, S. T., Moulds, M. and Guthrie, R. (2000) Treating acute distress disorder: an evaluation of cognitive behavior therapy and supportive counseling techniques. American Journal of Psychiatry 157 (11): 1889-91.

Fauerbach, J. A., Lawrence, J., Haythornthwaite, J., Richter, D., McGuire, M., Schmidt, C. and Munster, A. (1997) Preburn psychiatric history affects posttrauma morbidity. Psychosomatics 38 (4): 374-85.

Fauerbach, J. A., Lawrence, J. W., Munster, A. M., Palombo, D. A. and Richter, D. (1999) Prolonged adjustment difficulties among those with acute posttrauma distress following burn injury. Journal of Behavioral Medicine 22 (4): 359-78.

Glenn, D. M., Beckham, J. C., Feldman, M. E., Kirby, A. C., Hertzberg, M. A. and Moore, S. D. (2002) Violence and hostility among families of Vietnam veterans with combat related posttraumatic stress disorder. Violence and victims 17 (4): 473-89.

Gonsalkorale, W. M. (1996) The use of hypnosis in medicine: the possible pathways involved. European Journal of Gastroenterology and Hepatology 8(6): 520-4.

McFarlane, A. C. and Papay, P. (1992) Multiple diagnosis in posttraumatic stress disorder in the victims of a natural disaster. Journal of Nervous and Mental Disorders 180 (8): 498-504.

Miller, A. C., Hickman, L. C. and Lemasters, G. K. (1992) A distraction technique for control of burn pain. Journal of Burn Care and Rehabilitation 13 (5): 576-580.

Resnick, H. S., Kilpatrick, D. J., Dansley, B. S., Saunders, B. E. and Best, C. L. (1993) Prevalence of civilian trauma and posttraumatic stress disorder in a representative national sample of women. Journal of Consulting Clinical Psychologists 61 (6): 984-91.

Riva, G. (1997) In: Virtual Reality in Neuro-Psycho-Physiology. (edited by G. Riva) pp. 1-209. IOS Press, Ohmsha, ISBN:90 5199 364 1.

Schrader, K. A. (1996) Stress and Immunity after traumatic injury: the mind-body link. AACN Clinical Issues 7(3): 351-358.

Simms, L. J., Watson, D. and Doebbeling, B. N (2002) Confirmatory factor analysis of posttraumatic stress symptoms in deployed and non-deployed veterans of the gulf war. Journal of Abnormal Psychology 111 (4): 37-47.

Taal, L. and Faber, A. W. (1998) Posttraumatic stress and maladjustment among adult burn survivors 1 to 2 years postburn. Part II: the interview data. Burns 24 (5): 399-405.

Zach, G. A. (1980) Hypnosis, Part I: A historical overview. Compendium of Continued Education in Dentistry 11(5): 291-297.

1. A method for inducing hypnosis in a patient in a partially immersive virtual reality environment comprising subjecting said patient to verbal suggestion and visual images shown by a two or three dimensional display device.

2. A method according to claim 1 further comprising subjecting said patient to additional auditory stimuli.

3. A method according to claim 1 wherein the two or three dimensional display device is a wide field-of-view display.

4. A method according to claim 3 wherein the wide field-of-view display is a projection screen.

5. A method according to claim 1 wherein the two or three dimensional display device is a head mounted display.

6. A method for inducing hypnosis in a patient in a fully immersive virtual reality environment comprising subjecting said patient to verbal suggestion and visual images shown by a two or three dimensional display device.

7. A method according to claim 6 further comprising subjecting said patient to additional auditory stimuli.

8. A method according to claim 6 wherein the two or three dimensional display device is a wide field-of-view display.

9. A method according to claim 8 wherein the wide field-of-view display is a projection screen.

10. A method according to claim 6 wherein the two or three dimensional display device is a head mounted display.

11. A method according to claim 1 in the treatment of a psychosomatic disease in a human.

12. A method according to claim 11 wherein the psychosomatic disease is irritable bowel syndrome, herpes simplex, headache or stomach cramps.

13. A method according to claim 1 in the treatment of a somatic disease in a human.

14. A method according to claim 13 wherein the somatic disease is irritable bowel disease, arthritis, multiple sclerosis or cancer.

15. A method according to claim 1 in the treatment of a behavioural disorder in a human.

16. A method according to claim 15 wherein the behavioural disorder is attention deficit disorder, attention deficit hyperactivity disorder, post trauma distress disorder, postnatal depression, bulimia or obesity.

17. A method according to claim 1 in the treatment of a phobia in a human.

18. A method according to claim 17 wherein the phobia is fear of dogs, spiders, snakes, flying, heights or ships.

19. A method according to claim 1 in palliative care.

20. A method according to claim 19 wherein the palliative care is used to treat grief or fear of death.

21. A method according to claim 1 in the treatment of trauma or physical injury in humans.

22. A method according to claim 21 wherein the trauma or physical injury is burns, post surgical wounds, puncture wounds or ulcers.

23. A method according to claim 1 in the treatment of mood disorders in humans.

24. A method according to claim 23 wherein the mood disorder is anxiety, panic disorders or depression.

25. A method according to claim 1 in the treatment of and/or management of pain in humans.

26. A method according to claim 1 with or without chemical agents in anaesthetizing a human for surgical procedures.

27. A method according to claim 1 to alleviate boredom.

28. A method according to claim 1 with or without chemical agents to assist with childbirth.

29. A method according to claim 1 to improve skill development in sport, work or any other physical endeavour.

30. A method according to claim 1 to motivate humans involved in sport, work, war or any other endeavour that requires motivational modification.
